(19) United States
(12) Patent Application Publication
(10) Pub. No.: US 2005/0222640 A1
(43) Pub. Date: Oct. 6, 2005

(54) Heart muscle stimulator and pacing method for treating hypertension

(76) Inventors: Robert S. Schwartz, Rochester, MN (US); Robert Van Tassel, Excelsior, MN (US)

Correspondence Address: BECK AND TYSVER P.L.L.C., 2900 THOMAS AVENUE SOUTH, SUITE 100, MINNEAPOLIS, MN, 55416, US

(21) Appl. No.: 11/057,279

(22) Filed: Feb. 11, 2005

Related U.S. Application Data
(60) Provisional application No. 60/544,112, filed on Feb. 12, 2004.

(51) Int. Cl. A61N 1/18; A61N 1/362

(52) U.S. Cl. 607/44

(57) ABSTRACT

The method calls for the periodic electrical stimulation of the heart muscle to alter the ejection profile of the heart thus reducing the observed blood pressure of the patient. The therapy may be invoked by an implantable blood pressure sensor associated with a pacemaker like device.

### CROSS REFERENCE TO RELATED APPLICATIONS

The present case is the utility conversion of U.S. Provisional Application 60/544,112 filed Feb. 12, 2004 entitled “Antihypertensive Cardiac Pacing” which is incorporated herein in its entirety. Applicants claim the benefit of the earlier filing date of the provisional application for all that it contains and teaches.

### FIELD OF THE INVENTION

The present invention relates generally to stimulating or pacing the human heart and more particularly to a device and method for treating hypertension through a periodic or episodic electrical stimulation of selected portions of the heart at selected times.

### BACKGROUND OF THE INVENTION

The device of present invention is a heart muscle stimulator for supplying an electrical stimulation therapy to anatomic structures of the heart for altering blood pressure. The device and method can be used to treat hypertension and other disorders. The invention is disclosed in the context of treating high blood pressure with or without congestive heart failure (CHF).

Hypertension is a very common medical condition, with increasing prevalence with older age groups. The most common form of the disorder is termed ‘essential hypertension’, and it is a condition that may result from a mismatch between the heart output and the resistance of the major blood vessels. Untreated or poorly controlled hypertension can result in CHF. CHF is a common disease with a large number of clinical associations. The invention is expected to be a significant advance in the treatment of hypertension and CHF, and other medical complications of hypertension including but not limited to kidney failure, atherosclerosis and vascular disease, heart attack, and stroke. The method and device can be used to treat other diseases and the selected indications that are used as examples and should be considered illustrative and not limiting.

It has become a common practice to treat some instances of heart failure with pacemaker resynchronization therapy. This therapy has been shown to reduce mortality and improve the quality of life in patients with progressive congestive heart failure. Several pacing modalities have been adopted including biventricular pacing where leads are fixed to pace in both the right ventricle (RV) and the left ventricle (LV). Conventional multisite pacing can be used for resynchronization therapy by providing electrical stimuli at two sites in the same chamber with a fixed time delay. In general, the timing between pacing pulses is selected to “optimize” the “heartbeat”. Typically, success of resynchronization therapy is determined by an increase in the ejection fraction or cardiac output of the heart over the “normal” or background output in response to the same demand. In most instances of resynchronization therapy, the patient's blood pressure or hypertensive state is not explicitly taken into account during the prescription of the pacing parameters.

In many instances a CHF patient undergoing chronic resynchronization therapy will be taking appropriate drugs such as ACE inhibitors, Angiotensin receptor blockers, diuretics, beta blockers, digoxin, other inotropes, and antiarrhythmics.

In general the most widely used therapy to control blood pressure alone or in conjunction with other disease conditions is through systemic administration of drugs.

Other device based approaches for reducing blood pressure through pacing are known. For example, device based therapies include pacemaker type stimulators for non-cardiac structures for treating hypertension as taught by U.S. Pat. No. 6,073,048 to Kieval which discloses a device that delivers stimulation to arterial baroreceptors to lower systemic blood pressure indirectly through neurogenically mediated pathways.

Pacemakers that incorporate pressure sensors are known from U.S. Pat. No. 6,522,926 to Kieval which shows a pacemaker for optimizing the AV delay interval of a patient's heart to increase cardiac output.

### SUMMARY OF THE INVENTION

In stark contrast to conventional resynchronization therapy the present invention attempts to induce a controlled and temporary “lack of coordination” between mechanical and electrical activities of parts of the heart. This will lower blood pressure (BP) through changing the energy and power coupling between the heart and the major blood vessels. Applicants adopt “modified or modulated synchrony” to describe the intended effect.

When used to treat hypertension, the present invention acts to promote or cause myocardial dyssynchronization which has the beneficial clinical effect of reducing observed blood pressure through alteration of the activation profile of the heart in both spatial and temporal dimensions. This therapy also alters the resulting ejection profile as observed in the time domain or alternatively spatial across the anatomic structures of the heart. The result of this therapy is to reduce pretreatment blood pressure to a clinically beneficial post treatment value. The preferred stimulation regime may or may not result in a beneficial alteration of the contractility profile of the heart. The impact of the therapy on contractility is unknown at this time. Conceptually this technique offers direct stimulation of the heart to effect its endpoints, rather than relying on secondary effects of drugs and the like as known in the prior art.

### BRIEF DESCRIPTION OF THE DRAWINGS

Through the several figures like reference numerals identify identical structure wherein:

FIG. 1A is a VOO pacing configuration used in an experiment;

FIG. 1B is a data graph of blood pressure reduction at three pacing rates as measured in connection with an experiment;

FIG. 2A is a VOO pacing configuration used in an experiment;

FIG. 2B is a data graph of blood pressure reduction at three pacing rates;

FIG. 3A is a DOO pacing configuration used in an experiment;

FIG. 3B is a data graph of blood pressure reduction at three pacing rates;

FIG. 4A is a biventricular DOO pacing configuration used in an experiment;

FIG. 4B is a data graph of blood pressure reduction at three pacing rates;

FIG. 5A is a DOO pacing configuration used in an experiment;

FIG. 5B is a data graph of blood pressure reduction at three pacing rates;

FIG. 6A is a multisite VOO pacing configuration used in an experiment;

FIG. 6B is a data graph of blood pressure reduction at three pacing rates;

FIG. 7A is a multisite VOO pacing configuration used in an experiment;

FIG. 7B is a data graph of blood pressure reduction at three pacing rates;

FIG. 8 is a diagram showing an exemplary and illustrative heart stimulator capable of carrying out the invention;

FIG. 9 is a diagram comparing drug therapy with the inventive therapy; and,

FIG. 10 is a diagram showing how the inventive stimulation can be combined into a hybrid stimulator/drug therapy; and,

FIG. 11 is a diagram summarizing pacing configurations.

### DESCRIPTION OF THE INVENTION

### DEFINITIONS

Some terms are not consistently used with precision in the medical literature. For this reason and for the purposes of interpreting this document the flowing definitions obtain:

Dyssynchrony is inducing a cardiac ejection cycle where the normal spatial contraction sequence is altered, either within a chamber or across multiple cardiac chambers. It may also refer to changes in contraction within a chamber or across multiple chambers in time. This means that the ejection of blood may for example be delayed, or prolonged.

Hypertension is defined as blood pressure systolic greater than 130 mmHg and/or diastolic greater than 90 mmHg.

Altered Contractility Profile is any disturbance of cardiac contraction that changes the power or energy of the heart. It is best measured by Emax from the end systolic pressure-volume loop relationship across multiple different loading conditions.

Pre Treatment Contractility Profile is the spatial and temporal contraction of individual and combined heart chambers prior to treatment. Contractility is best measured by Emax from the end systolic pressure-volume loop relationship across multiple different loading conditions.

Altered Ejection Profile is any disturbance of cardiac contraction, either within a chamber or across multiple chambers, that alters the resulting blood pressure as a bolus of blood is ejected from the heart.

Pre Treatment Ejection Profile is the spatial and temporal contraction of individual and combined heart chambers prior to treatment.

Congestive heart failure (CHF) is the name given to a spectrum of clinical symptoms. Usually the heart is enlarged and has an inability to sufficiently supply the body's blood pressure and flow needs without generating abnormal intracardiac blood pressures and/or flows.

Overview

In general terms, the inventive method is the intentional reduction of a patient's blood pressure though a cardiac stimulation regime that modifies the synchrony between or within the chambers of the heart. In the simplest embodiments which form illustrative but not limiting descriptions of the invention, pacing level stimuli are applied to the heart trough fixed leads of conventional design. The location of the leads or the timing of the stimuli is selected to alter the ejection profile or the contractility profile of that heartbeat. This modification or modulation of synchrony lowers blood pressure.

The preferred device is intended to deliver pacing level stimuli to the heart muscle to treat hypertension. In general the proposed and preferred device will monitor blood pressure with an indwelling blood pressure sensor and invoke a modulated synchrony therapy that results in blood pressure reduction. Experimental data and computer modeling verify that this therapy may be used alone or in conjunction with drug therapy.

A blood pressure (BP) transducer will be exposed to systolic, diastolic, and indeed continuous blood pressures and the device may compute a mean pressure for a beat or several beats of the heart. The BP data may also be used to compute dP/dt and other BP measures. In most examples the existence of hypertension is taken as a fixed BP threshold. However this threshold may vary as a function of time of day or measured activity. In essence the threshold used to invoke the therapy may itself vary.

The modified therapy may be invoked on demand in response to a BP threshold. Alternatively or in addition the therapy may be provided on a periodic (circadian) basis, or even on a beat-by-beat interval, for example skipping one or more beats. It may also be based on the coincidence of a threshold BP occurring simultaneously with measured activity. In some embodiments the therapy may be initiated by the patient or the physician on an acute basis. It is expected that the therapy will not be continuous, but it will be chronic, throughout the lifetime of a hypertensive patient.

Many drugs are traditionally used for hypertension. These include ACE inhibitors, Angiotensin Receptor blockers (ARB blockers), diuretics, beta receptor blockers, alpha receptor blockers, vasodilators, calcium channel blockers, centrally mediated antihypertensives such as methyl-DOPA, and others. The proposed therapy will enhance the antihypertensive effects of these drugs, allowing them to work more effectively. The therapy can be adjusted to modulate the hypertensive effects of these drugs.

In many hypertensive patients, blood pressure may be reduced by the administration of a drug that widens the QRS complex by dispersing the electrical-myocardial conduction and contraction that may be additive with the therapy. Candidate drugs include Tricyclic antidepressants, neuroleptics lithium procanimide lidocaine and derivatives, Class I antiarrhyythmics, salbutamol, flecainide, sertindole, propofenone, amiodarone and others.

### Illustrative Embodiments and Associated Experiments

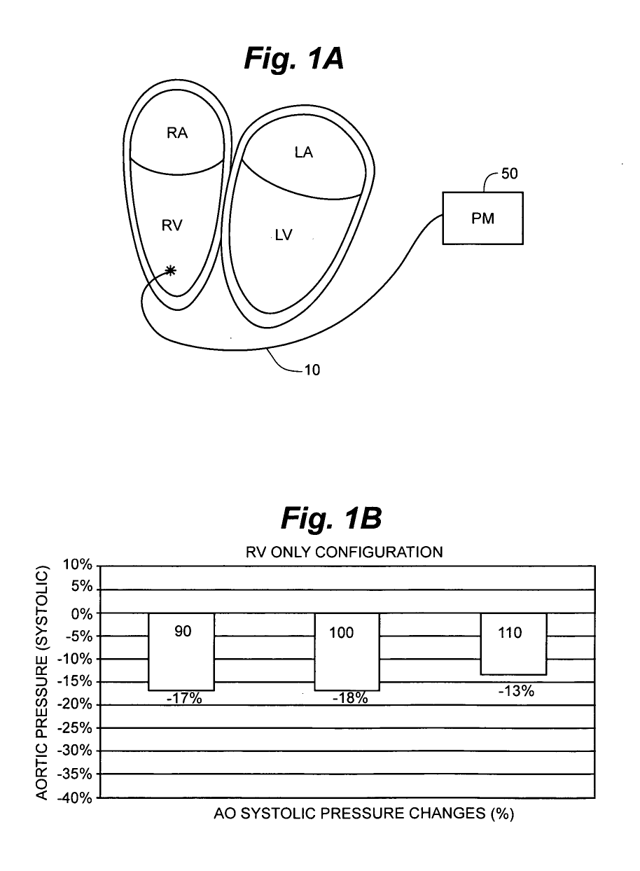

FIG. 1 through FIG. 7 are intended to show stimulation configurations that can be used to carry out or promote dyssynchrony between and within cardiac chambers to control blood pressure. Panel A of each figure shows the lead configuration and panel B shows the measured blood pressure reduction from a control measurement made in the same animal in normal sinus rhythm under otherwise similar conditions. Each panel of the data is taken at progressively higher pacing rates to capture the heart.

Thus in each instance the control for the experiment is taken in the same animal. The pre-treatment activation profile or prêt-treatment contractility profile corresponds to the BP in sinus rhythm. In a similar fashion the pre-treatment ejection profile corresponds to the BP in sinus rhythm.

FIG. 1A shows a lead 10 located in the apex of the RV coupled to a pacemaker 50 (PM). Capturing the heart at pacing rates of 90, 100, and 110 BPM results in the data shows in the graph of FIG. 1B. In this figure a reduction of BP by 16 percent is shown with no observable rate dependence.

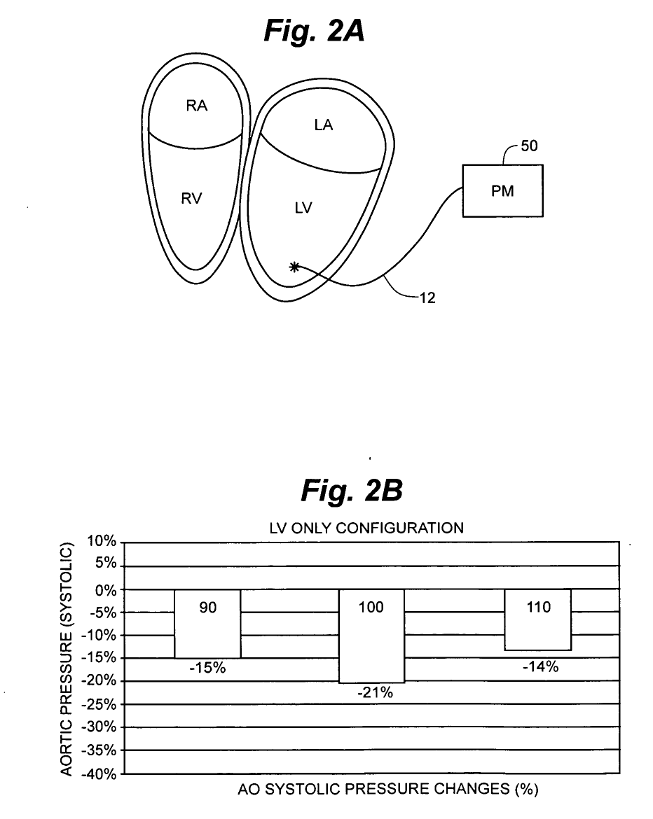

FIG. 2A shows a lead 12 located the apex of the LV with a VVI pacing configuration operating effectively in a VOO modality with pacemaker 50. The several pacing rates seen the graph of FIG. 2B show a −17% BP reduction without rate dependence.

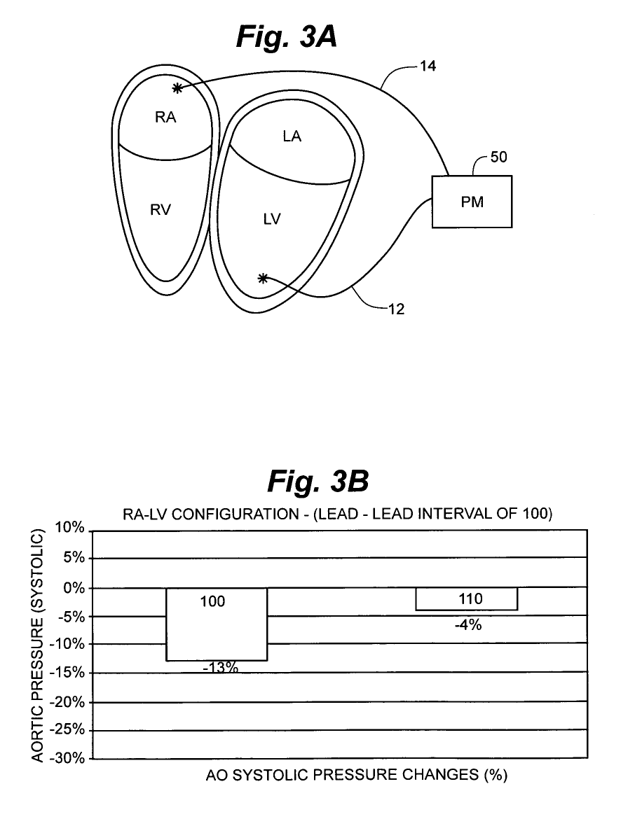

FIG. 3A shows a DOO modality where the right atrium is paced by lead 14 at a rate above sinus rhythm by pacemaker 50. The LV is paced through lead 12 after a variously short A-V delay preventing normal sinus conduction and contractility. FIG. 3B shows that a −8% BP reduction was achieved without observable dependence on the AV interval scan.

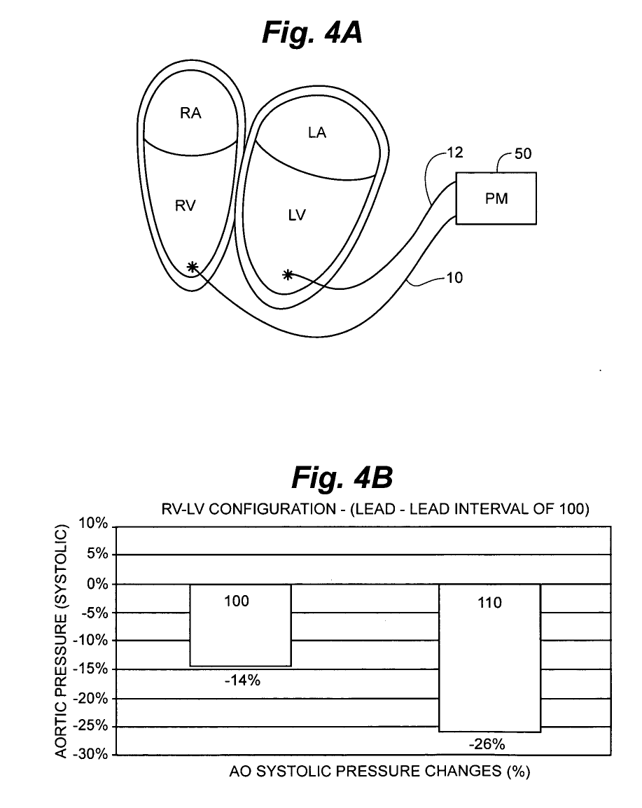

FIG. 4A shows a biventricular modality with VOO pacing of both the RV and the LV through leads 10 and 12. A progressive change was made to the RV-LV pacing interval. The RR interval was above sinus rhythm and scanned as well. No discernable dependence on rate was observed however a large −20% BP reduction was observed as seen in FIG. 4B.

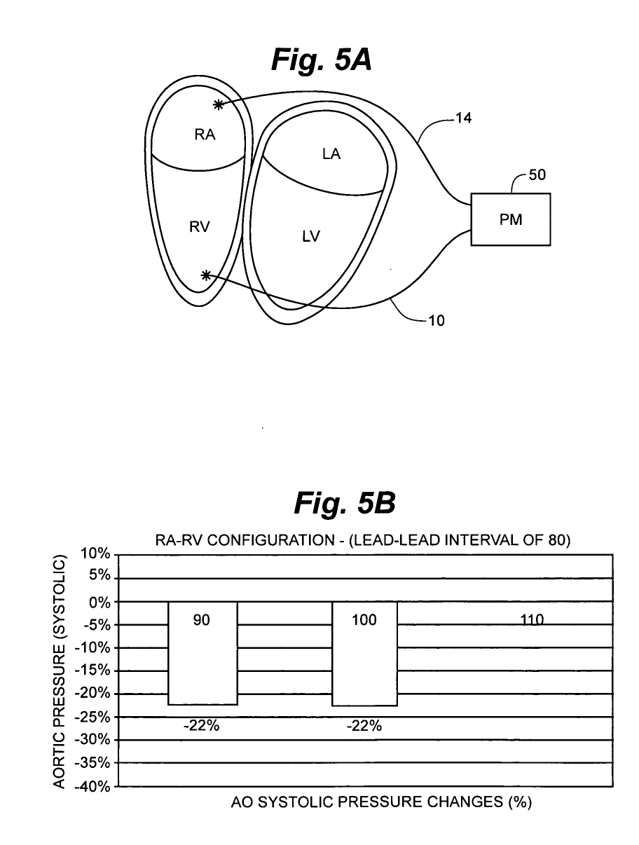

FIG. 5A shows a simple DOO pacing regime carried out in DDD mode. The AV delay was varied from 20 to 80 milliseconds and a marked reduction of BP −22% was observed as depicted in FIG. 5B.

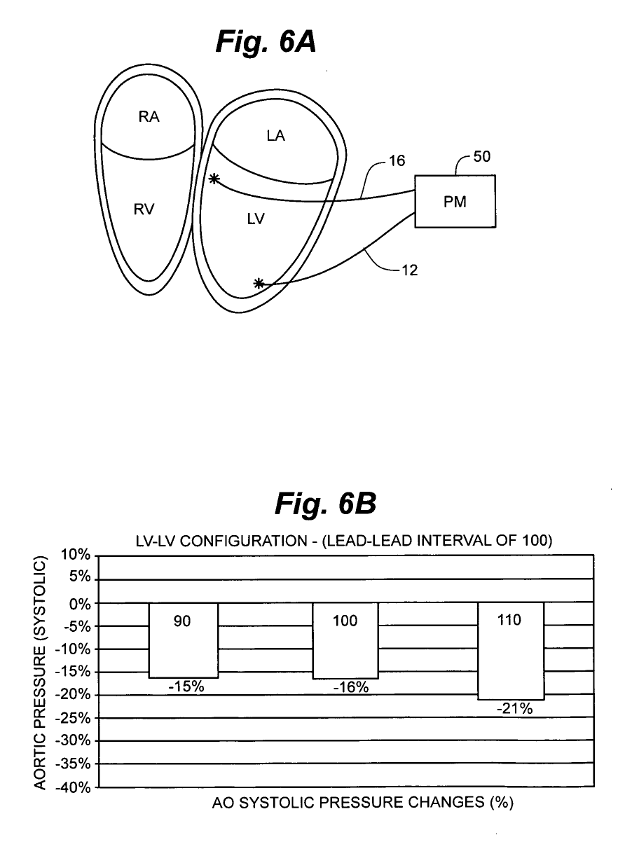

FIG. 6A shows a lead 10 in the LV at a first position and a second lead 12 located in the same chamber along the septum wall. The Lva-Lvb time interval was varied and FIG. 6B shows the −17% BP reduction achieved with this protocol.

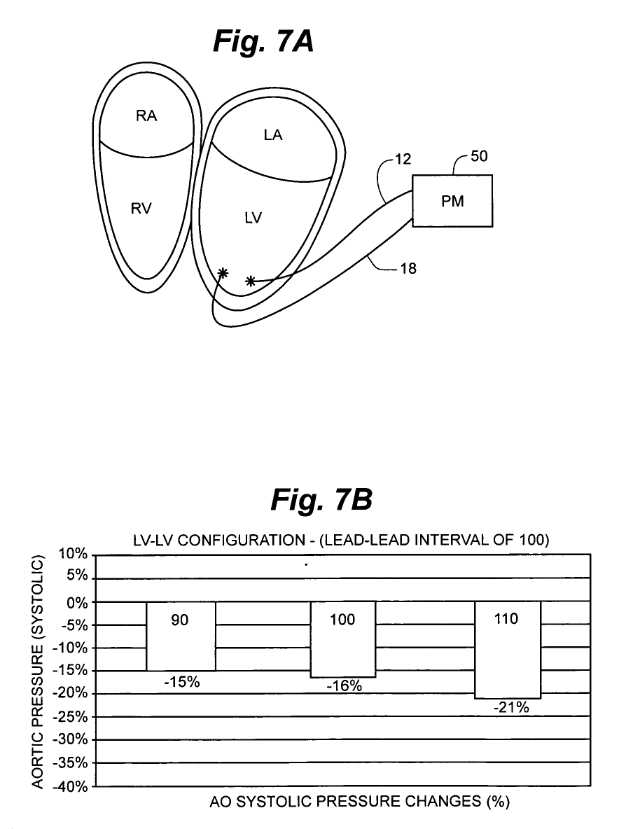

FIG. 7A shows an intraventricular anterior-inferior placement of leads 10 and 12. Burst pacing to 300 BPM showed a BP reduction of −10% as seen in FIG. 7B.

FIG. 9 reflects additional computer modeling work was performed to evaluate the effect of modified synchrony pacing or stimulation protocols in comparison to a more conventional drug therapy.

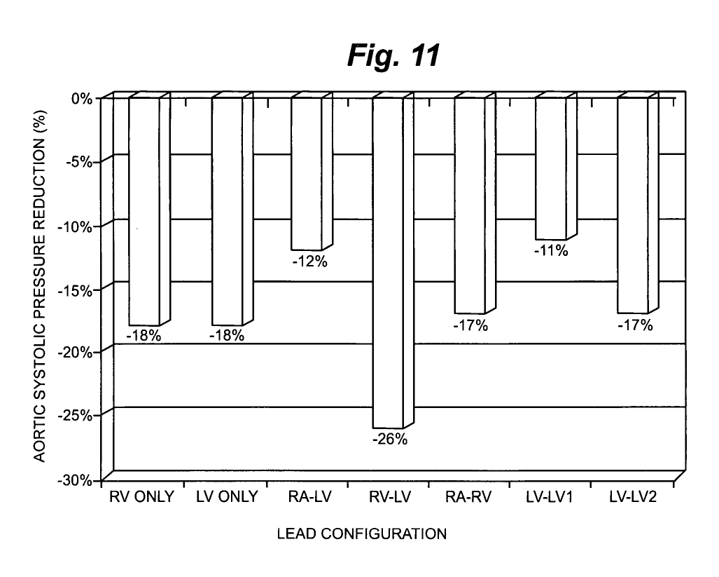

FIG. 11 is a chart that summarizes the percent reduction based upon pacing configurations used in the experiment.

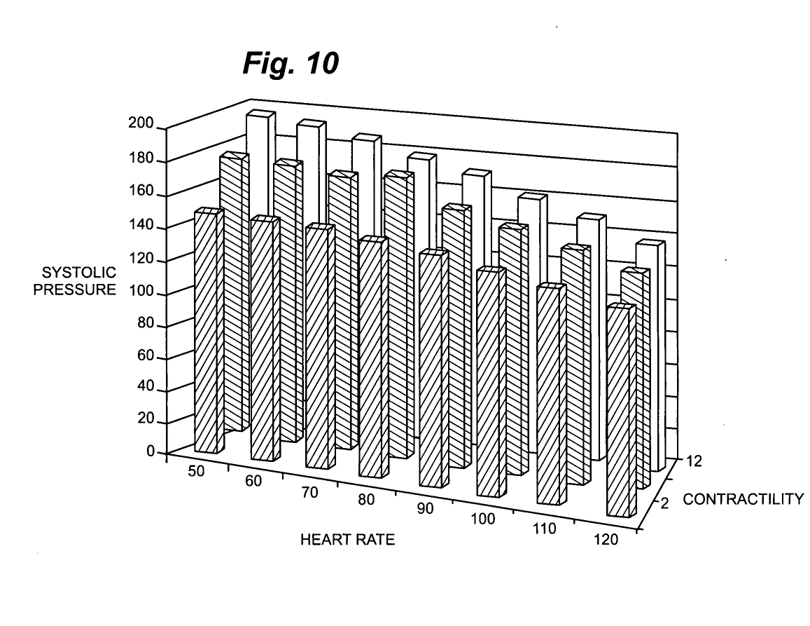

FIG. 10 reflects additional computer experimenting showing the value of a combined drug and stimulation therapy. In the figure at a lower than normal does of contractility reducing drug the percent change in BP reduction as a function of pacing increases dramatically. It is expected that combination therapy will be effective as well where the device therapy takes place in a patient with a “background” dose of the antihypertensive drug.

Interpretation and Benefits

FIGS. 1 through FIG. 7 illustrate that any number of conventional stimulation regimes or therapies can be invoked to modify synchrony within or between the heart chambers. The best therapy may vary from patient to patient and some experimentation will be required to tailor a device for a patient. Based on the experiment it appears that the greatest reduction in BP is achieved with RV-LV dyssynchrony stimulation. As seen in FIG. 7A and 7B.

However it should be clear that the time the stimulus is delivered or the location of the stimulus can used to achieve the beneficial modification of synchrony independent of lead location.

FIG. 9 shows the relationship between the inventive cardiac stimulation and more traditional pharmacology on the control of blood pressure. Line 100 is the identity line corresponding to no therapy and the pre treatment and post treatment blood pressure is the “same”. The pharmacology line 110 shows the control of blood pressure by a drug alone. For example, a patient having a pre treatment pressure of 200 mm of Hg is reduced to about 150 mm of Hg with a hypothetical drug. The linearity of the response however shows that the patient with an acceptable pretreatment BP of 100 mm of Hg would experience a drop to an undesirable BP of approximately 80 mm of Hg with the same drug. This treatment line shows that the systemic and chronic treatment of BP with drug can have an undesirable but concomitant effect on BP.

The device therapy is seen on line 120 which offers a BP reduction therapy which is modest and proportional to the need for therapy. The highly nonlinear behaviors of BP reduction with the inventive stimulation regime is of benefit to the patient since it brings a greater percent reduction benefit at the higher more pathologic BP values. Of considerable benefit is the fact the BP reduction occurs quickly with the onset of the stimulation regime and diminishes slowly when the stimulation is discontinued. It is preferred to have the therapy invoked when a threshold is exceeded and then continue for a fixed period of time for example 1 hour then the therapy stops. Activity monitors or real time clocks may be used as well.

Hardware Implementation

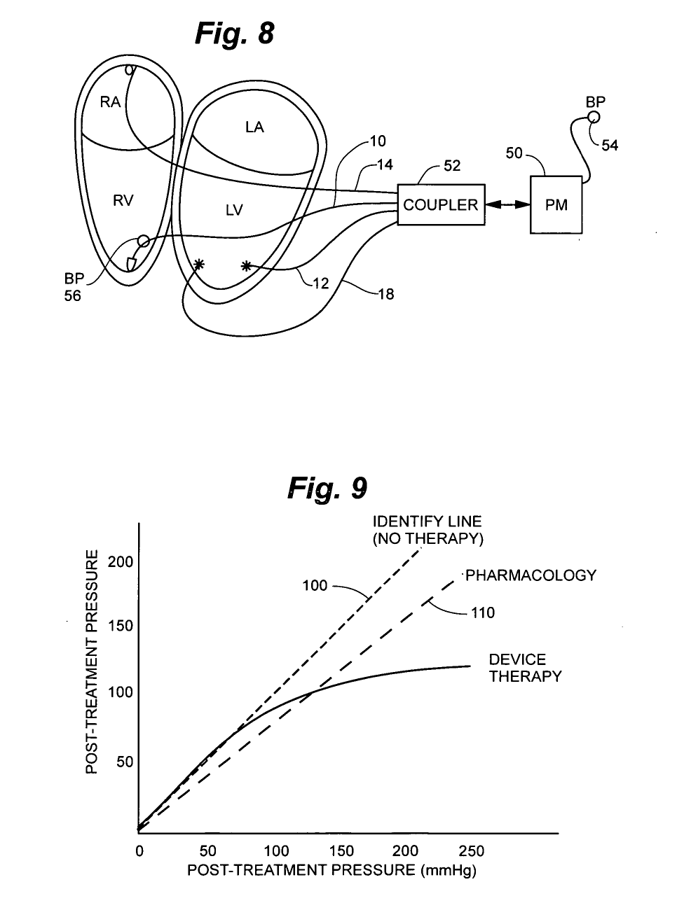

A representative but not limiting embodiment of a pacing device 50 to carry out the invention is shown in FIG. 8. The drawing shows a conventional heart stimulator capable of delivering heart stimulation to leads implanted at various locations in the heart. A connection block 52 allows selection of lead configurations as set forth in FIGS. 1 through 7. Typically only a subset of the leads shown in the figure are required for carrying out the therapy. Both sensing and pacing can occur at each lead location in the heart. All rates and timing intervals are available in the heart stimulator. A blood pressure sensor is located on a lead. Typical locations are in the RV or remotely in other regions of the vasculature. In another configuration, left ventricular cavity pressure can be measured with a device placed in the right ventricle that penetrates the ventricular septum and emerges into the left ventricular cavity. This device may also have a pressure transducer that lies within the septal wall, and measures intra-septal force as a surrogate for contractility.

A blood pressure transducer 54 is located on either a separate blood pressure lead or as a separate sensor 56 on a ventricular lead 10. It is important to note that other blood pressure transduction devices may be incorporated into the device. Although BP measurement is preferred other BP proxy measurements may be substituted within the scope of the invention.

A blood pressure transducer is provided to measure blood pressure to determine the existence of hypertension. The blood pressure monitoring transducer may be located on a lead for example the RV ventricular lead or a separate BP lead may be provided.

It is expected that a BP algorithm will be developed which provides a BP threshold. The threshold may vary with time of day or patient activity. Once detected the stimulator will delivery a therapy for a treatment time. It is expected that the treatment time will be selected by the physician and it may be terminated automatically or it may time out. This episodic therapy may be used alone or in conjunction with a drug regime.

Proposed Mechanism of Action

It is believed that the present invention induces a controlled and temporary “inefficiency” in the mechanical function of the heart. This inefficiency is produced and controlled by altering either or all, the normal pacing rate, the normal electrical path of ionic gradient flow through the heart, or dyssynchronization between the right and left ventricles. In the normal heart, initiation of the heart beat occurs in the sinoatrial node that resides towards the epicardial surface of the right atrium close to the junction of the superior vena cava. Nodal cells have a constantly changing resting membrane potential measured in respect to the voltage difference between the outside and inside of the cell. There are protein channels that traverse the cardiac pacemaker cell membrane and allow ionic currents to flow across the membrane depending on channel opening and the diffusion gradient of various ions such as sodium, potassium and calcium. In the pacemaker cells, there are sodium and calcium channels that increase pacing rate by decreasing their resistance to ion flow from the outside to inside of the cell based on their diffusion gradients. These ions carry a positive charge thereby inducing a decrease in the resting membrane potential and make the cell less negative. As this process continues in time, the cell membrane reaches an activation voltage potential whereby the calcium channel opens completely, the doubly positively charged calcium ions flow into the cell causing a complete depolarization. This depolarization then conducts three dimensionally throughout the atrial contractile cells. Contractile cells differ from pacemaker cells in that they maintain a stable resting membrane potential by allowing a controlled amount of potassium ions to leave the cell, determined by the membrane potential. They also differ in that when they are confronted with either a positively charged depolarization wavefront or an artificially induced electrical stimulus, a sodium channel, instead of a calcium channel, is activated and the cell becomes depolarized. The depolarization in a contractile muscle cell then allows calcium ions to be release intracellularly from the sarcoplasmic reticulum and a cell contraction occurs.

When the depolarization wavefront of positive charges reaches the atrioventricular node, those cells become depolarized and the unidirectional wavefront continues down the “bundle of his” to the apex of the ventricles. Purkinje fibers rapidly conduct this depolarization wavefront away from the apex and into the muscle cells of the ventricles leading towards the base of the heart. The natural pathway of electrical conduction from the apex towards the base also results in a slight spiraling pathway. This allows the ventricular muscle to effectively and efficiently “wring” out blood from the chambers.

By implanting electrical stimulating leads in the ventricular chambers, the present invention allows for an artificial activation of the ventricular multidirectional depolarization wavefront. If the electrical stimulation leads are placed in the apex of the ventricles, a close approximation of the natural pathway of electrical-mechanical coupling occurs. If the pacing rate however is overdriven higher than the normal pacing rate, there will be less time for filling of blood into the chambers driven by the venous side filling pressure. In accordance with Starling's Law, less blood filling the chamber results in less stretch on the actin and myosin contractile filaments, and therefore less contractile force developed to eject blood from the chambers. Less ejection volume and ventricular pressure consequently results in less systemic blood pressure developed.

This invention also allows for de-synchronizing the right and left ventricular chambers. The stimulation leads may be placed in one or both of the ventricular apices and stimulated in a fashion that allows one chamber to contract prior to the other. Because the right ventricle anatomically wraps around the left ventricle and produces a chamber containing part of the left ventricle wall, a dyssynchronous contraction between the right and left chambers results in an inefficiency in mechanical function and resultant ejection of blood, initially from the right ventricle that results in less filling in the left ventricle and less ejection and lowered systemic blood pressure. Another aspect to this invention is the deliberate activation of single or multiple pacing sites in the ventricle (s) at locations other than the apex. Initiation of contraction at sites towards the base of the chamber results in myocardial contraction forces being applied to intra-chamber retrograde movement of blood and static pressure development in the apical part of the chamber. This force can be directly subtracted from the overall force developed by the ventricle to ejecting blood into the systemic circulation, resulting in lowered blood pressure.

1. A method, carried out with an implanted heart muscle stimulator, for treating blood pressure disorders in a patient comprising the steps of:
measuring the patient's pre treatment blood pressure (BP), with an external or implanted sensor, corresponding to both a pretreatment cardiac ejection profile and a pretreatment cardiac contractility profile;
comparing the measured pre treatment blood pressure with a treatment threshold;
stimulating the patient's heart with an electrical stimulus at one or more times and/or locations to alter the cardiac ejection profile thereby reducing the measured blood pressure from the pretreatment blood pressure value.

2. The method of claim 1 wherein the stimulating step comprises:
placing a lead in the RV right ventricle of the heart and coupling the lead to said muscle stimulator.

3. The method of claim 1 wherein the stimulating step comprises:
placing a lead in the LV left ventricle of the heart and coupling the lead to said muscle stimulator.

4. The method of claim 1 wherein the stimulating step comprises:
placing a lead in the LV left ventricle of the heart and placing a lead in the RV right ventricle and coupling each of said leads to said muscle stimulator.

5. The method of claim 1 wherein the stimulating step comprises:
placing a first lead at a first location in the RV right ventricle of the heart and placing a second lead at a second location different from said first location in the RV right ventricle and coupling each of said leads to said muscle stimulator.

6. The method of claim 1 wherein the stimulating step comprises:
placing a first lead at a first location in the LV left ventricle of the heart and placing a second lead at a second location different from said first location in the LV left ventricle and coupling each of said leads to said muscle stimulator.

7. The method of claim 1 wherein the stimulating step comprises:
placing a first lead at a first location in the RA right atrium of the heart and placing a second lead at a second location in the LV left ventricle and coupling each of said leads to said muscle stimulator.

8. The method of claim 1 wherein the stimulating step comprises:
placing a first lead at a first location in the RA right atrium of the heart and placing a second lead at a second location in the RV right ventricle and coupling each of said leads to said muscle stimulator.
